SEQUENCE LISTING (2 sequences)

SEQ ID NO: 1 - DNA, 19 bp, Artificial
  5′ sense primer for amplifying PDF gene
cattcaaaagaccgacacc

SEQ ID NO: 2 - DNA, 18 bp, Artificial
  3′ antisense primer for amplifying PDF gene
aggtgcacagtggaagga

(19) United States
(12) Patent Application Publication
(10) Pub. No.: US 2004/0253207 A1
(43) Pub. Date: Dec. 16, 2004

(54) Methods and compositions for prostate epithelial cell differentiation

(75) Inventors: Keith A. Hruska, St. Louis, MO (US); Lala R. Chaudhary, Ballwin, MO (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(73) Assignee: Barnes-Jewish Hospital

(21) Appl. No.: 10/704,506

(22) Filed: Nov. 7, 2003

Related U.S. Application Data
(60) Provisional application No. 60/424,948, filed on Nov. 8, 2002.

(51) Int. Cl. A61K 38/18

(52) U.S. Cl. 424/85.1; 514/12

(57) ABSTRACT

Methods and compositions for treating prostate cancer by promoting prostate epithelial cell differentiation are described. Treatment methods involve administration of an active form of prostate-derived factor (PDF), or of an inactive PDF precursor, or of a combination of a proprotein convertase (PC) with a PDF precursor, to increase the biological activity of PDF in the subject and promote prostate epithelial cell differentiation.

### CROSS-REFERENCE TO RELATED APPLICATIONS

This application claims priority to U.S. provisional application No. 60/424,948, filed Nov. 8, 2002, the specification of which is herein incorporated by reference in its entirety.

### FEDERALLY-SPONSORED RESEARCH OR DEVELOPMENT

Not Applicable.

### BACKGROUND OF THE INVENTION

The present invention relates in general to the field of cancer treatments, and in particular to regulation of prostate epithelial cell differentiation as a treatment for prostate cancer.

Prostate cancer is a malignant tumor that begins in the prostate gland of men. The prostate is a walnut-sized gland located behind the base of the penis, in front of the rectum and below the bladder. It surrounds the urethra, the tube-like channel that carries urine and semen through the penis. The prostate's main function is to produce seminal fluid, the liquid in semen that protects, supports and helps transport sperm. Over 95% of prostate cancers are adenocarcinomas, cancers that develop in glandular tissue. Another important type of prostate cancer is known as neuro-endocrine or small cell anaplastic cancer. This type tends to metastasize earlier, but does not produce prostate specific antigen (PSA).

Prostate cancer is the most common cancer among men. In 2002, approximately 189,000 new cases of prostate cancer are expected to be diagnosed in the United States. Prostate cancer is the second leading cause of cancer death in men (S. L. Parker et al., Cancer statistics, CA. Cancer J. Clin. 47:5-27 (1997)), and an estimated 30,200 deaths are expected to occur in 2002. Although the number of deaths from prostate cancer is declining among all men, the death rate remains more than twice as high in African-Americans as in Caucasians.

Eighty-three percent of all prostate cancer cases are discovered when the disease is limited to the prostate and surrounding organs. In these cases, 100% of patients are expected to live at least five years after diagnosis. The overall relative five-year survival rate for all stages of prostate cancer is 96%. The ten-year and fifteen-year survival rates are 75% and 54% respectively.

The current treatment options for prostate cancer include surgery, radiation, medical therapy, a combination of medical therapy and surgery or radiation, chemotherapy, and watchful waiting. A patient's treatment options will generally depend upon his age and the stage of the disease. However, these treatment options have numerous side effects and in many instances are not used to treat the neoplasia in its early stages of growth.

Certain members of the transforming growth factor-β (TGF-β) superfamily of proteins have been shown to affect differentiation and growth of prostate cancer cells. Bone morphogenetic proteins (BMP's) are expressed in normal rat and human prostate and prostate cancer cells. S. E. Harris et al., Prostate 24:204-211 (1994). TGFβ1, TGFβ2, and TGFβ3 are expressed in normal and malignant human prostate. K. T. Perry et al., Prostate 33:133-140 (1997). TGF-β1 has been linked to tumorigenesis of the prostate. P. Wikstrom et al., Role of transforming growth factor-β1 in prostate cancer, Microscopy Res. Tech. 52:4111-419 (2001).

Proteins in the TGF-β superfamily, once secreted, must be activated to have biological effects. TGF-B proteins are first synthesized as larger biologically-inactive precursors (also called pro-proteins) that are proteolytically processed at a dibasic site (R-X-X-R) to release mature, active TGF-βs. The processing site is a consensus cleavage motif for proprotein convertases such as furin. However, currently very little else is known about secretion and processing of TGF-βs in prostate cells.

Prostate-derived factor (PDF) is a divergent member of TGF-β superfamily proteins that is highly expressed in the placenta and the prostate and is also involved in the differentiation of the prostate epithelium. PDF is also known as PLAB (R. Hromas et al., Biochem. Biophys. Acta. 1354:40-44 (1997)), placental transforming growth factor-β, (PTGF-β) (M. Yokoyama-Kobayashi et al., J. Biochem. 122:622-626 (1997)), macrophage inhibitory cytokine-1 (MIC-1) (M. R. Bootcov et al., Proc. Nat'l Acad. Sci. USA 94:11514-11519 (1997)), and growth and differentiation factor-15 (GDF-15) (M. Bottner et al., Gene 237:105-111 (1999)).

PDF is expressed in normal and malignant prostate cells. V. M. Paralkar et al., J. Biol. Chem. 273:13760-13767 (1998); R. Thomas et al., Int. J. Cancer 93:47-52 (2001). The PDF gene is down-regulated in primary prostate cancer tissue compared to non-neoplastic prostate tissue, but re-appears in secondary metastatic lesions in bone and in lymph nodes. R. Thomas et al., (2001). Differential synthesis and secretion of PDF by various prostate cancer cells have not previously been examined. Thus, the precise nature of PDF secretion and processing and its relationship to tumorigenesis in the prostate remains unclear.

### BRIEF SUMMARY OF THE INVENTION

The present invention provides methods and compositions for the treatment of prostate cancer and the regulation of prostate epithelial cell differentiation. In particular, the present invention provides a cell-differentiation therapy using active PDF, PDF precursors and proprotein convertases, alone or in combination, to promote expression and activity of PDF in prostate cancer cells. Embodiments of the invention include administering an active form of PDF to cancerous cells having a receptor for PDF and administering a proprotein convertase (PC) to promote the production of active PDF in cells that have the ability to secrete pro-protein PDF but do not process it or process it inefficiently.

In an exemplary embodiment, a method for treating prostate cancer in a subject in need thereof comprises promoting prostate epithelial cell differentiation by administering to the subject an agent for increasing the biological activity of PDF in the subject. The agent is, for example, a therapeutically effective amount of active PDF. Alternatively the agent is an amount of a precursor of PDF and an amount of proprotein convertase wherein together the amount of the precursor of PDF and the amount of proprotein convertase are sufficient to provide a therapeutically effective amount of active PDF. Alternatively the agent is a therapeutically effective amount of proprotein convertase.

In another embodiment, a method for treating prostate cancer in a subject in need thereof, comprises obtaining a sample of prostate tissue from the subject, characterizing cancerous cells in the tissue sample to determine whether the cells possess a receptor for PDF, characterizing the cells to determine whether the cells synthesize and secrete a precursor of PDF, and characterizing the cells to determine whether the cells process the precursor of PDF to produce active PDF. In one alternative embodiment of this method, wherein the cells do not synthesize and secrete the precursor of PDF, the method further comprises administering to the subject a therapeutically effective amount of active PDF. In another alternative embodiment of this method, wherein the cells do not synthesize and secrete the precursor of PDF, the method further comprises the step of administering to the subject an amount of the precursor of PDF together with an amount of proprotein convertase wherein together the amount of the precursor of PDF and the amount of proprotein convertase are sufficient to provide a therapeutically effective amount of active PDF. In still another alternative embodiment of this method, wherein the cells synthesize and secrete the precursor of PDF but do not process the precursor of PDF, the method further comprises the step of administering to the subject a therapeutically effective amount of active PDF. In yet another alternative embodiment of this method, wherein the cells synthesize and secrete a precursor of PDF but do not process the precursor of PDF, the method further comprises the step of administering to the subject a therapeutically effective dose of a proprotein convertase for processing the precursor of PDF.

In another embodiment, a method for treating prostate cancer in a subject in need thereof comprises promoting prostate cell differentiation in the subject by administering to the subject an agent for increasing the biological activity of PDF in the subject at an early stage of the prostate cancer.

In another embodiment, a composition for treating or preventing prostate cancer in a subject comprises a therapeutically effective amount of active PDF in a pharmaceutically acceptable carrier.

In another embodiment, a composition for treating or preventing prostate cancer in a subject comprises an amount of an inactive precursor of PDF and an amount of proprotein convertase in a pharmaceutically acceptable carrier, wherein together the amount of the inactive precursor of PDF and the amount of proprotein convertase are sufficient to provide a therapeutically effective amount of active PDF.

In another embodiment, a composition for treating or preventing prostate cancer in a subject comprises a therapeutically effective amount of proprotein convertase in a pharmaceutically acceptable carrier.

Other features of the present invention will be in part apparent to those skilled in the art and in part pointed out in the detailed description below.

### BRIEF DESCRIPTION OF THE FIGURES

These and other features, aspects and advantages of the present invention will become better understood with regard to the following description, appended claims and accompanying figures where:

FIG. 1A shows the RT-PCR analysis of prostate-derived factor mRNA expression;

FIG. 1B shows an immunoblot with anti-pro-PDF antibody and anti-PDF antisera demonstrating differential PDF synthesis and secretion by different human prostate cancer cells;

FIG. 2 shows an assay of PC activity determined by measuring the cleavage of fluorogenic substrate, boc-RVRR-amc, in human prostate cancer cell lines and the effect of 100 μM CMK;

FIG. 3 shows an immunoblot with anti-pro-PDF antibody and anti-PDF antisera demonstration inhibition of PDF processing by CoM in LNCaP cells;

FIG. 4 shows an immunoblot with anti-cytokeratin 8, 14, 18 and 19 antibodies demonstrating the effects of CMK on prostate epithelial cell differentiation markers; and

FIG. 5 shows an immunoblot with anti-PSA antibody demonstrating the effect of CMK on the DHT-stimulated PSA expression in LNCaP cells.

### DETAILED DESCRIPTION OF THE INVENTION

Generally, the nomenclature used hereafter, and the laboratory procedures are those well known and commonly employed in the art. Unless defined otherwise, all technical and scientific terms used herein have the same meaning as commonly understood by one of ordinary skill in the art to which this invention relates. Although any methods and materials similar or equivalent to those described herein can be used in the practice or testing of the present invention without deviating from the scope or purpose of the invention, the preferred methods and materials are described.

The cell-differentiation therapy of the present invention is based in part upon the discovery of differential production, secretion and processing of PDF in various prostate cancer cell lines including, but not limited to, LNCaP, PC3 and DU145. The cell-differentiation therapy of the present invention is also based in part upon the discovery of proprotein convertase-dependent alterations of prostate epithelial differentiation as assessed by cytokeratin-expression patterns and androgen-dependent prostate-specific antigen (PSA) production.

As used herein, “prostate-derived factor” or “PDF” (V. M. Paralkar et al., J. Biol. Chem. 273:13760-13767 (1998)) refers to all species and homologs of PDF including PLAB (R. Hromas et al., Biochem. Biophys. Acta. 1354:40-44 (1997)), PTGF-β (M. Yokoyama-Kobayashi et al., J. Biochem. 122:622-626 (1997)), MIC-1 (M. R. Bootcov et al., Proc. Nat'l Acad. Sci. USA 94:11514-11519 (1997)) and GDF-15 (GDF-15) (M. Bottner et al., Gene 237:105-111 (1999)), as well as recombinant PDF proteins, and functional derivatives of PDF, PLAB, PTGF-β, MIC-1 and GDF-15.

As used interchangeably herein, “prostate-derived factor precursor”, “PDF precursor”, “precursor of PDF” and “pro-PDF” refer to the inactive PDF proprotein, which is a secreted, larger and immature form of PDF that must first be proteolytically processed by a proprotein convertase to the mature, biologically active PDF.

As used interchangeably herein, “proprotein convertases” and “PCs” include, but are not limited to, furin, PC1/3, PC2, PACE4, PC4, PC5/6, BMP1 and PC7/8, as well as functional derivatives and homologs thereof, and recombinant PCs which act to regulate PDF in mammals.

As used herein, a “recombinant PDF protein” is a protein which is obtained through the use of recombinant nucleic acid technology. Such recombinant protein's primary structure may be identical to that of its naturally-occurring counterpart PDF or may contain additional or different amino acid residues including single or several mutations.

As used herein, a “recombinant PC” is a proprotein convertase which is obtained through the use of recombinant nucleic acid technology. Such recombinant proprotein convertase's primary structure may be identical to that of its naturally-occurring proprotein convertase counterpart or may contain additional or different amino acid residues, including single or several mutations.

As used herein, a protein which is a “functional derivative” of PDF, PLAB, PTGF-β, MIC-1, GDF-15 is a protein which possesses structural and functional similarity to PDF. Structurally similar proteins include, for example, proteins differing from PDF by amino acid residue deletions, insertions, or conservative substitutions which do not substantially diminish the function of promoting cell differentiation.

As used herein, a proprotein convertase which is a “functional derivative” of a PC is a proprotein convertase which possesses structural and functional similarity to a PC. Structurally similar proprotein convertases include, for example, proprotein convertases differing from a PC by amino acid residue deletions, insertions, or conservative substitutions which do not substantially diminish the function of promoting PDF expression and/or cell differentiation.

As used herein, “pharmaceutically-acceptable carriers” are well known to those skilled in the art such as phosphate buffer or saline. Such pharmaceutically-acceptable carriers may be aqueous or non-aqueous solutions, suspensions, and emulsions. Examples of non-aqueous solvents are propylene glycol, polyethylene glycol, vegetable oils, and injectable organic esters such as ethyl oleate. Aqueous carriers include water, alcoholic/water solutions, emulsions or suspensions, including saline and buffered media. Parenteral vehicles include sodium chloride solution, Ringer's dextrose, and the like. Preservatives and other additives may also be present, such as, for example, antimicrobials, antioxidants, chelating agents, inert gases and the like.

As used herein, “subject” means any animal or artificially-modified animal capable of developing or sustaining prostate cancer. Artificially-modified animals include, but are not limited to, mice, rats, dogs, guinea pigs, ferrets, rabbits, and primates. In the preferred embodiment, the subject is human.

As used herein, “administering” may be effected or performed using any of the various methods known to those skilled in the art. The administering may comprises administering intravenously, intramuscularly and subcutaneously.

As used herein, a “Therapeutically-effective dose” is a dose to selectively inhibit the proliferation of prostate cancer cells in an afflicted subject. Dosage levels are highly dependent on the nature of the disease or situation, the condition of the patient, the judgment of the practitioner, and the frequency and mode of administration.

As used herein, “cell differentiation” means the sum of the processes whereby cells mature and attain their mature adult form and function, for example, the differentiation of basal epithelial cells to luminal secretory cells.

As used herein, the terms “treating” or “to treat” means to alleviate symptoms, eliminate the causation either on a temporary or permanent basis, or to prevent or slow the appearance of symptoms. The term “treatment” includes alleviation, elimination of causation of or prevention of prostate cancer.

As used herein with respect to PDF, the term “biological activity” refers to the biochemical behavior of pro-PDF and PDF in vivo, through which PDF ultimately renders its effects on body tissues. “Biological activity” is intended to encompass synthesis of pro-PDF, secretion of pro-PDF, and processing of pro-PDF to the mature, active PDF form by proprotein convertase.

As used herein with respect to the biological activity of PDF, the terms “increase” and “increasing” refer to enhancing or at least partially restoring the down-regulation of biological activity of PDF that is observed in cancerous prostate tissue, as described in the Example herein.

As used herein with respect to prostate cancer, the term “early stage” refers to prostate cancer that demonstrates limited development in terms of tumor burden in the prostate, such that a tumor mass is not detectable on digital rectal exam (DRE), but cancerous prostate tissue is detectable through microscopic evidence such as measurement of biomarkers such as prostate-specific antigen (PSA). Methods such as PSA doubling time (PSADT) and PSA rates of increase (PSA velocity or PSAV) are known in the art and are used to detect prostate cancer before a mass is detectable by DRE.

In an exemplary embodiment, a method for treating prostate cancer in a subject in need thereof comprises promoting prostate epithelial cell differentiation in the subject by administering to the subject an agent for increasing the biological activity of PDF in the subject . The agent is, for example, a therapeutically effective amount of active PDF. Alternatively, the agent is an amount of precursor of PDF and an amount of proprotein convertase wherein together the amount of the precursor of PDF and the amount of proprotein convertase are sufficient to provide a therapeutically effective amount of active PDF. Alternatively, the agent is an amount of proprotein convertase. Any of these agents may be administered in the form of a pharmaceutical composition containing including the agent together with a pharmaceutically acceptable carrier.

The choice of agent to administer will depend in part on an evaluation of the factors contributing to the down-regulation of PDF activity in the prostate tissue of the subject. The down-regulation may result from a down-regulation of PDF receptor expression in the cells, or may result from down-regulation of synthesis and secretion of pro-PDF, or may result from down-regulation of processing of pro-PDF to the mature, active PDF form, or may result from some combination of all of these factors. To distinguish among these possibilities, a sample of prostate tissue is obtained, and cancerous cells in the tissue sample are characterized to determine first whether the cells possess a receptor for PDF, which indicates that the cells will be responsive to exogenous PDF therapy. The cells are further characterized to determine whether the cells synthesize and secrete a precursor of PDF, and characterized to determine whether the cells process the precursor of PDF to produce active PDF. If the cells do not synthesize and secrete the precursor of PDF, then in one embodiment, a therapeutically effective amount of the mature, active PDF is administered to the subject. Alternatively, an amount of a precursor of PDF and an amount of proprotein convertase together are administered to the subject. If the cells synthesize and secrete pro-PDF, but do not process pro-PDF to the mature, active PDF, then in one embodiment a therapeutically effective amount of the proprotein convertase is administered to the subject so that the subject's pro-PDF can be processed.

The methods are intended to encompass administration of an amount of a precursor of PDF and an amount of proprotein convertase together in a single dosage form, and also intended to encompass administration of the amount of the precursor of PDF in a pharmaceutically acceptable carrier in a first dosage form, and administering the amount of proprotein convertase in a pharmaceutically acceptable carrier in a second dosage form separate from the first dosage form.

In another embodiment, a method for preventing development of prostate cancer in a subject in need thereof comprises promoting prostate cell differentiation in the subject by administering to the subject an agent for increasing the biological activity of PDF in the subject at an early stage of the prostate cancer. The agent is a therapeutically effective amount of active PDF, a therapeutically effective amount of proprotein convertase, or an amount of pro-PDF and an amount of proprotein convertase wherein together the amount of pro-PDF and the amount of proprotein convertase are sufficient to provide a therapeutically effective amount of active PDF. The pro-PDF and proprotein convertase can be administered together in single dosage form in a pharmaceutically acceptable carrier, or can be administered with the amount of the PDF in a pharmaceutically acceptable carrier in a first dosage form, and the amount of proprotein convertase in a pharmaceutically acceptable carrier in a second dosage form separate from the first dosage form.

When PDF, pro-PDF or proprotein convertase, alone or in combination, are provided along with a pharmaceutically acceptable carrier, novel compositions for the treatment of prostate cancer are formed. For use for treatment of animal subjects, the compositions of the invention can be formulated as pharmaceutical or veterinary compositions. Depending on the subject to be treated, the mode of administration, and the type of treatment desired, e.g., prevention, prophylaxis, therapy; the compositions are formulated in ways consonant with these parameters. A summary of such techniques is found, for example, in Remington's Pharmaceutical Sciences, latest edition, Mack Publishing Co., Easton, Pa. It should be understood that, for example, that the amount of PDF, or the combined amount of a pro-PDF and a proprotein convertase that is required to achieve the desired biological effect depends on a number of factors, including the specific individual compound or compounds chosen, the specific use, the route of administration, the clinical condition of the subject, and the age, weight, gender, and diet of the subject.

Novel compositions for treating prostate cancer include, for example, a composition including an amount of active PDF together in a pharmaceutically acceptable carrier. In an alternative embodiment, a composition for treating prostate cancer includes an amount of an inactive precursor of PDF and an amount of proprotein convertase in a pharmaceutically acceptable carrier, wherein together the amount of the inactive precursor of PDF and the amount of proprotein convertase are sufficient to provide a therapeutically effective amount of active PDF. The amount of inactive precursor of PDF and an amount of proprotein convertase may be combined together in a pharmaceutically acceptable carrier in a single dosage form, or formulated in two separate dosage forms such as two separate injectable solutions, or two separate tablets or capsules. Alternatively, a composition for treating prostate cancer includes an amount of proprotein convertase and a pharmaceutically acceptable carrier.

The administration of the compositions of the present invention may be pharmacokinetically and pharmacodynamically controlled by calibrating various parameters of administration, including the frequency, dosage, duration mode and route of administration. Variations in the dosage, duration and mode of administration may also be manipulated to produce the activity required.

In defining the use of a pro-PDF in combination with a proprotein convertase, the methods are intended to embrace administration of each agent in a sequential manner in a regimen that will provide beneficial effects of the combination, and is intended as well to embrace co-administration of these agents in a substantially simultaneous manner, such as in a single capsule or dosage device having a fixed ratio of these active agents or in multiple, separate capsules or separate dosage devices for each agent, where the separate capsules or dosage devices can be taken together contemporaneously, or taken within a period of time sufficient to receive a beneficial effect from both of the constituent agents of the combination.

Pharmaceutically acceptable carriers include, but are not limited to, physiological saline, Ringer's, phosphate solution or buffer, buffered saline, and other carriers known in the art. Pharmaceutical compositions may also include stabilizers, anti-oxidants, colorants, and diluents. Pharmaceutically acceptable carriers and additives are chosen such that side effects from the pharmaceutical compound are minimized and the performance of the compound is not canceled or inhibited to such an extent that treatment is ineffective.

The pharmaceutical compositions may be administered enterally and parenterally. Parenteral administration includes subcutaneous, intramuscular, intrasternal, intradermal, intramammary, intravenous, by infusion and other administrative methods known in the art. Enteral administration includes solution, tablets, sustained release capsules, enteric coated capsules, and syrups. When administered, the pharmaceutical composition may be at or near body temperature.

The subject combinations can be administered in the form of sterile injectable aqueous or olagenous suspensions. Such suspensions may be formulated according to the known art using those suitable dispersing of wetting agents and suspending agents which have been mentioned above, or other acceptable agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, n-3 polyunsaturated fatty acids may find use in the preparation of injectables.

The subject combinations can be administered orally, for example, as tablets, coated tablets, dragees, troches, lozenges, aqueous or oily suspensions, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of sweetening agents, flavoring agents, coloring agents and preserving agents in order to provide pharmaceutically elegant and palatable preparations.

Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, maize starch, or alginic acid; binding agents, for example starch, gelatin or acacia, and lubricating agents, for example magnesium stearate, stearic acid or talc. The tablets may be uncoated or they may be coated by known techniques to delay disintegration and adsorption in the gastrointestinal tract and thereby provide a sustained action over a longer period. For example, a time delay material such as glyceryl monostearate or glyceryl distearate may be employed.

Hard gelatin capsules contain the active ingredients admixed with an inert solid diluent, for example, calcium carbonate, calcium phosphate or kaolin, or as soft gelatin capsules wherein the active ingredients are present as such, or mixed with water or an oil medium, for example, peanut oil, liquid paraffin, or olive oil.

Aqueous suspensions can be produced that contain the active materials in admixture with excipients suitable for the manufacture of aqueous suspensions. Such excipients are suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate. The aqueous suspensions may also contain one or more preservatives, for example, ethyl or n-propyl p-hydroxybenzoate, one or more coloring agents, one or more flavoring agents, or one or more sweetening agents, such as sucrose or saccharin.

Oily suspensions may be formulated by suspending the active ingredients in an omega-3 fatty acid, a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents, such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavoring and coloring agents, may also be present.

Syrups and elixirs containing the novel combination may be formulated with sweetening agents, for example glycerol, sorbitol or sucrose. Such formulations may also contain a demulcent, a preservative and flavoring and coloring agents.

In a first embodiment of the present invention, undifferentiated prostate cancer cells are obtained from a subject and characterized to determine whether the cells possess a receptor for PDF. If so, an active form of PDF may be administered to the subject in order to promote differentiation in the cancer cells.

Alternatively, a PDF precursor may be administered to the subject followed by administration of a PC which processes the pro-PDF into PDF thereby promoting cell differentiation. However, cells without a receptor for PDF may not respond to such treatment. At present, this embodiment is less preferred as administration of two proteins to a subject may lead to an increased risk of an immunogenic response in and inconvenience to the subject.

In another alternative embodiment, the PDF precursor maybe a recombinant polypeptide engineered to provide unique interaction with a recombinant PC to produce active PDF or a functional homolog thereof. Such recombinant PDF precursors and PCs may comprise a native or mutant primary amino acid sequences, obtained by expression of a gene carried by a recombinant DNA molecule in a cell other than the cell in which that gene and/or protein is naturally found. In other words, the gene is heterologous to the host in which it is expressed and/or the subject to which it is administered. It should be noted that any alteration of a gene, including the addition of a polynucleotide encoding an affinity purification moiety to the gene, makes that gene unnatural for these purposes, and thus that gene cannot be ‘naturally’ found in any cell.

A recombinant DNA molecule encoding for such recombinant proteins may be defined either by its method of production or its structure. In reference to its method of production, e.g., a product made by a process, the process is use of recombinant nucleic acid techniques, e.g., involving human intervention in the nucleotide sequence, typically selection or production. Alternatively, it can be a nucleic acid made by generating a sequence comprising fusion of two fragments which are not naturally contiguous to each other, but is meant to exclude products of nature, e.g., naturally occurring mutants. Thus, for example, products made by transforming cells with any unnaturally occurring vector is encompassed, as are nucleic acids comprising sequences derived using any synthetic oligonucleotide process. Such is often done to replace a codon with a redundant codon encoding the same or a conservative amino acid, while typically introducing or removing a sequence recognition site. Alternatively, it is performed to join together nucleic acid segments of desired functions to generate a single genetic entity comprising a desired combination of functions not found in the commonly available natural forms. Restriction enzyme recognition sites are often the target of such artificial manipulations, but other site specific targets, e.g., promoters, DNA replication sites, regulation sequences, control sequences, or other useful features may be incorporated by design.

Such recombinant PDF precursors may be engineered to interact only with a recombinant PC. Thus, an increase in PDF production may be achieved while avoiding increased processing of other TGF-β members which may otherwise result from the administration of a non-recombinant PC. In addition, such recombinant proteins could aid in avoiding the processing of other members of the TGF-β super-family that might cause side effects.

Similar advantages may be achieved through the use of recombinant PCs which may be provided to specifically target natural pro-PDF. For example, effective recombinant PCs may result from manipulation of the natural PC's pro-PDF binding site or the PC's flanking sequences, thus adversely affecting the PCs ability to bind to and process non-PDF pro-proteins. Such recombinant PCs alone could yield advantages similar to those noted above regarding the recombinant pro-PDF and recombinant PC combination.

In a second embodiment, the subject's cancer cells are characterized to determine whether the cells are capable of secreting PDF but do not have the ability to process it or process it efficiently. If so, an active form of PDF may be administered or a PDF precursor followed by a therapeutically-effective dose of a proprotein convertase may be given to the subject to promote cell differentiation.

In a third embodiment, a therapeutically-effective dose of a PC in a pharmaceutically-acceptable carrier is administered to subjects having cancer cells with the ability to both secrete and process PDF. The proprotein convertase assists the cells in processing PDF thereby promoting cell differentiation.

The cell-differentiation therapy of the present invention is supported by the discovery that PDF is synthesized as a pro-PDF form in both LNCaP and PC3 prostate cancer cell lines. PDF is activated by proprotein convertases (PCs) in LNCaP, but not in PC3 cells. The differences in cell phenotypes of LNCaP and PC3 cells may be in part due to impaired maturation of the TGF-β superfamily members in PC3 cells. Prostate cancer cells including LNCaP and PC3 have been shown to produce a variety of BMPs which stimulate osteoblastic bone formation and cause osteoblastic metastasis (T. Yoneda, Cellular and molecular mechanisms of breast and prostate cancer metastasis to bone, Eur. J. Cancer 34:240-245 (1998)) Similarly, the mRNA expression of various BMPs has been reported in LNCaP and PC3 cells. In vivo, LNCaP tumors stimulate osteoblastic responses while PC3 tumors result in extensive bone destruction and osteolytic responses. (D. H. Shevrin et al., Development of skeletal metastasis by human prostate cancer in athymic nude mice, Clin. Exp. Metastasis 6:40-1-409 (1988); G. Soos et al., Comparative intraosseal growth of human prostate cancer cell lines LNCap and PC3 in nude mice, Anticancer Res. 17:4253-4258 (1997)). Differences in the activation of TGF-βs including PDF by PCs may contribute to these responses.

The present invention is further supported by the discovery that PCs, such as furin, exhibit different activity between human prostate cancer cell lines. In particular, the activity level of PCs is significantly higher in LNCaP cells as compared to DU145 and PC3 cells. Treatment of cells with decanoyl-Arg-Val-Lys-Arg-chloromethylketone (CMK), a synthetic protease inhibitor, inhibits activity of PCs in all cell lines. Thus, in accordance with the therapy of the present invention, PC activity in LNCaP cells is responsible for PDF processing. Although PC3 cells have low activity of PCs, PDF-processing activity is undetectable thereby suggesting that a specific PC active on PDF is deficient in PC3 cells Inhibition of PDF processing by CMK causes alterations in the regulation of luminal and basal prostate epithelial cell differentiation markers in prostate cancer. Addition of CMK in LNCaP cells results in the down regulation of cytokeratin 8,18 and 19 and the up regulation of cytokeratin 14 thereby indicating the loss of differentiated cell characteristics. It has recently been demonstrated that TGF-β induces the up regulation of luminal and down regulation of basal cytokeratins gene expression in rat normal prostate cell line (D. Danielpour, Transdifferentiation of NRP-152 rat prostatic basal epithelial cells toward a luminal phenotype: regulation by glucocorticoid, insulin-like growth factor-1 and transforming growth factor-beta, J. Cell. Sci. 112:69-179 (1999)). Similarly, in the present invention, the inhibition of PC activity reduces the processing of TGF-βs, including PDF, and results in the loss of differentiated cell phenotype. However, CMK treatment does not affect the expression of cytokeratins in DU 145 and PC3 cells. This may be due to the lower activity of PCs or absence of a specific PC.

In addition, CMK inhibits dihydrotestosterone-induced PSA expression. PSA is expressed in well-differentiated prostate epithelial cells and is regulated by androgens. (S. W. Hayward et al., The prostate: development and physiology, Radiol. Clin. North America 38:1-14 (2000)). Cooperation between androgen receptor and smad3 has been shown to induce PSA gene expression. (H. Y. Kang et al., From transforming growth factor-β signaling to androgen action: identification of smad3 as an androgen receptor coregulator in prostate cancer cells, Proc. Nat'l Acad. Sci. USA (2001)). The present invention is consistent with these observations. Thus, inhibition of processing of TGF-β superfamily proteins, including PDF, may result in the reduction of androgen-induced PSA expression.

Without further elaboration, it is believed that one skilled in the art can, using the preceding description, utilize the present invention to its fullest extent. The invention may be better understood, however, by reference to the Example which follows. The experiments detailed are to be construed as merely illustrative of the invention and do not limit the remainder of the disclosure or the scope of the invention in any way.

### EXAMPLE

Materials and Methods

CMK was obtained from Calbiochem (San Diego, Calif.) and dihydrotestosterone (DHT) was obtained from Sigma (St. Louis, Mo.). Anti-pro-PDF antibody, anti-Cytokeratin 8, 18, 19 antibodies, and anti-prostate specific antigen antibody were purchased from Santa Cruz Biotechnology (Santa Cruz, Calif.). Cytokeratin 14 and 15 from Chemicon (Temecula, Calif.). N-t-butoxycarbonyl-Arg-Val-Arg-Arg-7-amino-4-methylcoumarine (Boc-RVRR-AMC) was obtained from Bachem Bioscience (King of Prussia, Pa.). An antisera against mature PDF was raised by injecting a 20-aa peptide sequence (CQKTDTGVSLQTYDDLLAKD) located in the C-terminus of PDF to rabbits as described by Tan. (M. Tan et al., PTGF-β, a type β transforming growth factor (TGF-β) superfamily member, is a p53 target gene that inhibit tumor cell growth via TGF-β signaling pathway, Proc. Nat'l Acad. Sci. USA 97:109-114 (2000)). LNCaP, DU145 and PC3 cell lines were obtained from the American Type Culture Collection (Rockville, Md.). RPMI 1640 was purchased from Cellgro Mediatech (Herndon, Va.) and fetal bovine serum from Atlas Biologicals (Fort Collins, Colo.).

Cell Culture

Human prostate cancer cells were grown in RPMI 1640 supplemented with 10% fetal bovine serum and cultures maintained at 37° C. in a humidified atmosphere of 95% air and 5% CO₂.

Reverse Transcriptase-PCR Analysis

Total RNA was prepared from various cell lines using RNeasy kit (Qiagen, Valencia, Calif.) according to the manufacturer's instructions. cDNA was prepared using Superscript (Invitrogen, Carlsbad, Calif.). The efficiency of each cDNA reaction was assayed by amplification of β-actin transcripts with primers for β-actin (Promega, Madison, Wis.). Primers used to amplify PDF gene were: sense, 5′-CATTCAAAAGACCGACACC; antisense, AGGTGCACAGTGGAAGGA-3′. The PCR condition of PDF was as follows: 1 cycle at 94° C. for 3 min, 30 cycles at 95° C. for 15 sec, 54° C. for 30 sec, 72° C. for reaction used a volume of 50 μl with 2.0 mM MgCl₂ and 2.5 units of PLATINUM Taq DNA Polymerase (Invitrogen, Carlsbad, Calif.). To check the DNA contamination, control experiments in which no reverse transcriptase was added prior to the PCR were performed. Amplification products (10 μl) were separated by 8% polyacrylamide gel electrophoresis.

Western Blot Analysis

Cells (approximately 1×10⁶ cells) were seeded in 100 mm cell culture dishes and cultured in RPMI 1640 supplemented with 10% fetal bovine serum at 5% CO₂. Subconfluent cells were washed with phosphate buffered saline (PBS), lysed in lysis buffer (Cell Signaling Technology, Beverly, Mass.) and used for cell lysate. For the detection of PDF in conditioned media, subconfluent cells were serum-starved for 24 h. The serum free media were collected and precipitated with 10% trichloroacetic acid. After centrifugation, pellets were dissolved in the buffer which consisted of 8 M urea, 50 mM Tris (pH 8.0) and 0.1% NP-40. Samples were mixed in 5× Laemmli sample buffer with 2% beta-mercaptoethanol, subjected to electrophoresis on a 15% SDS-polyacrylamide gel, and transferred to PVDF membranes. Membranes were blocked with PBS containing 0.1% Tween 20 and 5% non-fat dry milk for 1 h at room temperature and incubated with the appropriate primary antibodies and antisera (1:500-2000) overnight at 4° C. After the membranes were washed three times with PBS containing 0.1% Tween 20, the membranes were incubated with appropriate horse-radish peroxidase-conjugate secondary antibodies (1:2000) for 1 h at 4° C. The protein bands were visualized by chemiluminescence using SuperSignal West Pico ECL kit (PIERCE, Rockford, Ill.). This protocol was used for the detection of PDF, cytokeratin and PSA.

Proprotein Convertase Activity Assay

For determination of cellular PC activity, 30,000 cells were seeded in 48-well plates for 24 hours. The next day, growth medium and CMK were added until a final concentration of 100 μM was reached. After a 24-hour incubation, media were replaced and cells were washed with PBS. Additional 48-well plates were prepared in the same manner for the determination of cell number which was used for normalization. 150 μl of assay medium which consists of RPMI 1640 with 0.25% Triton X-100 for permeabilization and boc-RVRR-amc (100 μM) as a fluorogenic substrate were added to each well. Fluorescence was measured at 360 nm excitation and 460 nm emission wavelengths after 4 h of substrate addition. The data were normalized to cell number. Human recombinant furin (Sigma, St. Louis, Mo.) was used for positive control.

Statistical Analysis

Data are presented as the mean ±SD of three culture wells in each of two to six independent trials. Statistical analysis was performed using Student's t test. P value <0.05 was considered significant.

Results

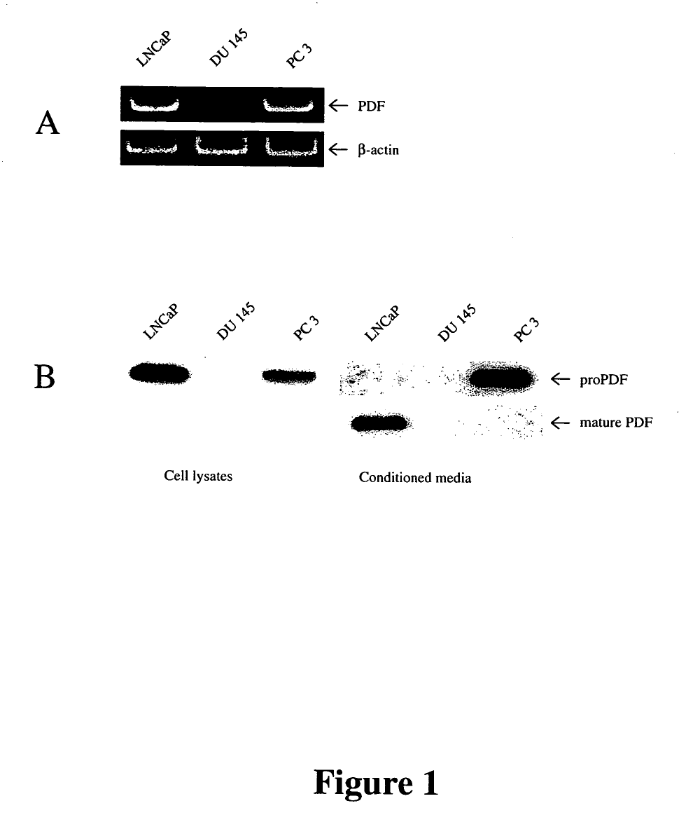

Cellular and secreted PDF were examined in different androgen-dependent (LNCaP) and androgen-independent (PC3 and DU145) cell lines. Total cellular RNA isolated from different prostate cancer cell cultures was subjected to RT-PCR. As shown in FIG. 1A, PDF-specific primers yielded a 100 bp fragment. Further, PDF mRNA expression was detected in LNCaP cells and PC3 cells, but not in DU145 cells. The presence of PDF protein was further confirmed by immunoblot analysis as shown in FIG. 1B. Pro-PDF was detected in LNCaP and PC3 cell lines by an anti-pro-PDF antibody (˜40 kd pro-PDF form) which recognized pro-PDF but not mature PDF. However, an anti-mature PDF antisera (˜17 kd mature PDF form) failed to detect mature PDF in cell lysates from all cell lines. Mature PDF, and not pro-PDF, was detected in LNCaP-conditioned media, but only pro-PDF was detected in PC3-conditioned media. These results demonstrate that mature PDF was secreted and processed only by LNCaP cells, but not by DU145 and PC3 cells. That pro-PDF was secreted by PC3 cells without processing suggest that these cells may be deficient in PCs. DU145 cells did not express PDF at all.

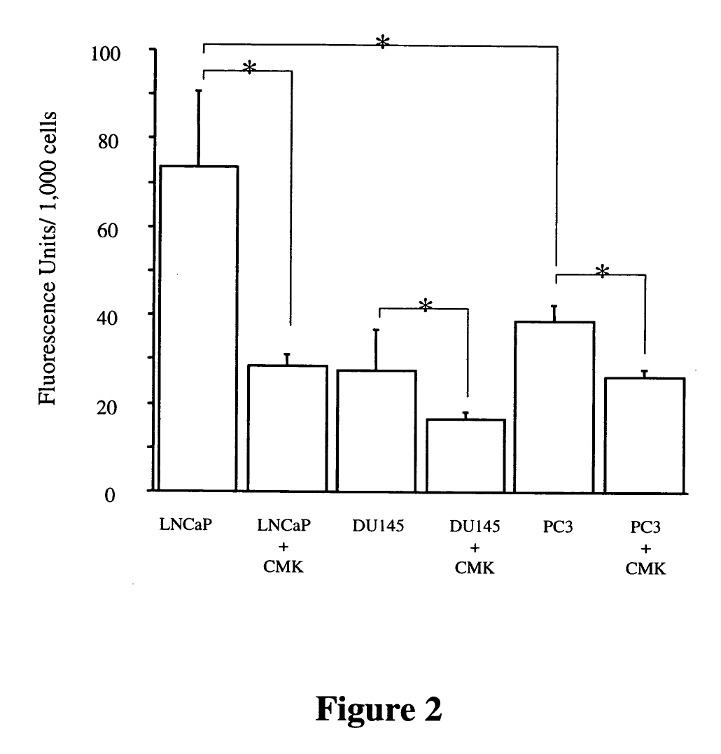

PC activity in prostate cancer cells and inhibition of PC activity by CMK was next examined. LNCaP, PC3 and DU 145 cells were incubated in the presence or absence of 100 μM CMK for 24 hours. Thereafter, 100 μM fluorogenic substrate, boc-RVRR-amc, was added and cells were incubated for an additional four hours. PC activity was assayed by measuring the cleavage of the fluorogenic substrate. The results shown in FIG. 2 demonstrate significantly higher PC activity in LNCaP cells than in PC3 and DU145 cells and the inhibition of PCs by CMK for 24 hours.

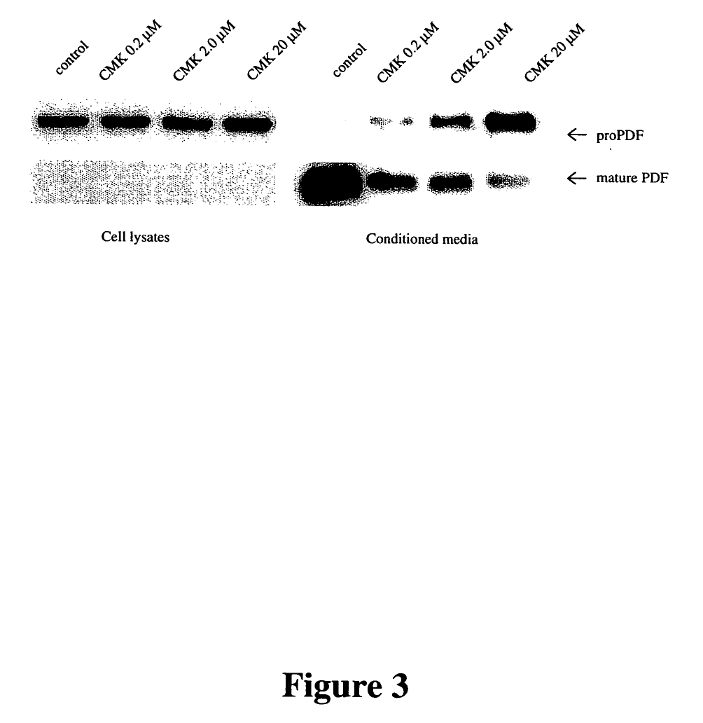

PC-dependent PDF processing in LNCaP cells was also examined in the presence of CMK by immunoblotting for PDF. Cells were cultured and serum-starved with and without various concentrations of CMK for 24 hours. Cells and supernatants were electrophoresed on 15% SDS-PAGE. Immunoblots were performed with anti-pro-PDF antibody (˜40 kd pro-PDF form) and anti-PDF antisera (˜17 kd mature PDF form). As shown in FIG. 3, the treatment with CMK resulted in the reduction of PDF processing in LNCaP-conditioned media in a dose-dependent manner, and concomitant increase in pro-PDF. CMK did not affect cellular PDF.

In the prostate, luminal epithelial cells differentiate from basal epithelial cells. The cell types are distinguished by the expression of cytokeratin 8 and 18 in the luminal and cytokeratin 5, 15, and 14 in the basal epithelial cells (Y. Xue et al., Identification of intermediate cell types by keratin expression in the developing human prostate, Prostate 34:292-301 (1998)). Cytokeratin 19 is suggested to be a marker of intermediate stage in the differentiation process of prostate cells (D. L. Hudson et al., Epithelial cell differentiation pathway in the human prostate: identification of intermediate phenotype by keratin expression. J. Histochem. Cytochem. 49:271-278 (2001)). Recent data have demonstrated that TGF-β induced the upregulation of luminal and the downregulation of basal cytokeratin gene expression in NRP-152 rat prostate basal epithelial cells. (D. Danielpour, Transdifferentiation of NRP-152 rat prostatic basal epithelial cells toward a luminal phenotype: regulation by glucocorticoid, insulin-like growth factor-1 and transforming growth factor-beta, J. Cell. Sci. 112:169-179 (1999)).

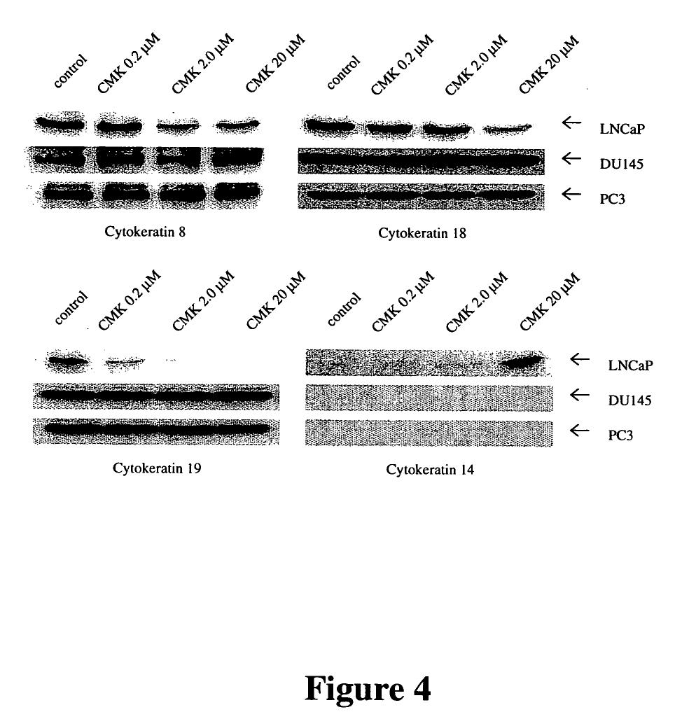

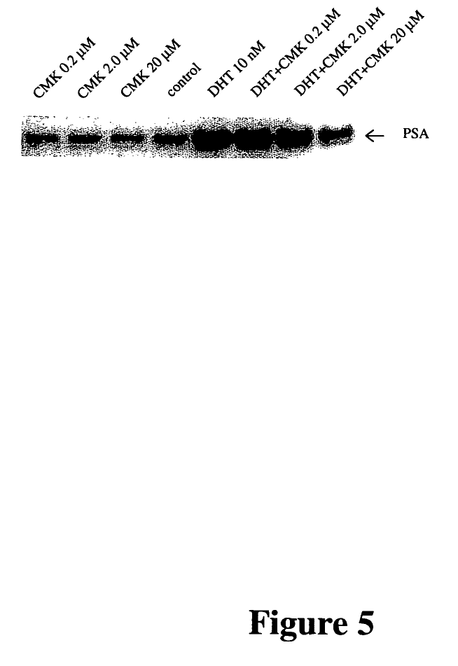

To examine whether CMK regulates the differentiated phenotype of LNCaP cells as a result of inactivation of TGF-β superfamily processing, including PDF, prostate cancer cell lines were treated with various concentrations of CMK in the presence of serum-free RPMI 1640. Culture medium and CMK were replaced every 24 hours. Cells were treated for 72 hours, lysed and electrophoresed on 15% SDS-PAGE. Immunoblots were performed with anti-cytokeratin 8 (˜52 kd), 14 (˜52 kd), 18 (˜45 kd) and 19 (˜44 kd) antibodies. Cytokeratin 8 and 18 represent luminal epithelia cell phenotype and cytokeratin 19 and 14 represent intermediate differentiation and basal epithelial cell phenotype, respectively. As shown in FIG. 4, CMK down-regulated the expression of cytokeratin 8, 18 and 19 in LNCaP cells, but not in DU145 and PC3 cells. Cytokeratin 14 was upregulated in a dose-dependent manner in LNCaP and was not detected in DU145 and PC3 cells.

PSA is a widely used and important serological marker for prostate cancer in patients. PSA expression is normally regulated by androgens in well-differentiated prostate epithelial cells. (S. W. Hayward et al., The prostate: development and physiology, Radiol. Clin. N. America 38:1-14 (2000)). Increased plasma TGF-β levels in the prostate cancer patients are correlated with elevated PSA levels (H. L. Adler et al., Elevated levels of circulating interleukin-6 and transforming growth factor-β1 in patients with matastatic prostatic carcinoma, J. Urol. 161:182-187 (1999)). A role of smad3, an intracellular signaling mediator of TGF-β, in the regulation of PSA gene expression via cooperation with androgen receptor has been reported in LNCaP cells. (H. Y. Kang et al., From transforming growth factor-β signaling to androgen action: identification of smad3 as an androgen receptor coregulator in prostate cancer cells, Proc. Nat'l. Acad. Sci. USA 98:3018-3023 (2001)). Therefore, it was important to investigate the effect of CMK on androgen-induced PSA expression to understand the mechanism of action of TGF-β superfamily, including PDF, in prostate tumorigenesis. LNCaP cells were treated with various concentrations of CMK for 24 hours in the presence or absence of DHT. Cell lysates were electrophoresed on 15% SDS-Page and then immunoblots were performed with anti-PSA antibody (˜37 kd). As shown in FIG. 4, androgen-induced PSA expression was inhibited by the addition of CMK, whereas the levels of endogenous PSA in the absence of DHT were not affected. Since PSA is not expressed in PC3 and DU145 cells, these experiments were not conducted in these cell types.

The detailed description set forth above is provided to aid those skilled in the art in practicing the present invention. Even so, this detailed description should not be construed to unduly limit the present invention as modifications and variation in the embodiments discussed herein can be made by those of ordinary skill in the art without departing from the spirit or scope of the present inventive discovery.

All publications, patents, patent applications and other references cited in this application are herein incorporated by reference in their entirety as if each individual publication, patent, patent application or other reference were specifically and individually indicated to be incorporated by reference.

What is claimed is:

1. A method for treating prostate cancer in a subject in need thereof, said method comprising promoting prostate epithelial cell differentiation in the subject by administering to the subject an agent for increasing the biological activity of PDF in the subject.

2. A method in accordance with claim 1 wherein administering to the subject an agent for increasing the biological activity of PDF in the subject comprises administering to the subject a therapeutically effective amount of PDF.

3. A method in accordance with claim 2 wherein administering to the subject a therapeutically effective amount of PDF comprises administering a therapeutically effective amount of PDF in a pharmaceutically acceptable carrier.

4. A method in accordance with claim 1 wherein administering to the subject an agent for increasing the biological activity of PDF in the subject comprises administering to the subject an amount of a precursor of PDF and an amount of proprotein convertase wherein together the amount of the precursor of PDF and the amount of proprotein convertase are sufficient to provide a therapeutically effective amount of PDF.

5. A method in accordance with claim 4 wherein administering an amount of a precursor of PDF and an amount of proprotein convertase comprises administering the amount of PDF and the amount of proprotein convertase together in single dosage form in a pharmaceutically acceptable carrier.

6. A method in accordance with claim 4 wherein administering an amount of a precursor of PDF and an amount of proprotein convertase comprises administering the amount of the precursor of PDF in a pharmaceutically acceptable carrier in a first dosage form, and administering the amount of proprotein convertase in a pharmaceutically acceptable carrier in a second dosage form separate from the first dosage form.

7. A method in accordance with claim 1 wherein administering to the subject an agent for increasing the biological activity of PDF in the subject comprises administering to the subject a therapeutically effective amount of proprotein convertase.

8. A method in accordance with claim 7 wherein administering a therapeutically effective amount of a proprotein convertase comprises administering a therapeutically effective amount of the proprotein convertase in a pharmaceutically acceptable carrier.

9. A method for treating prostate cancer in subject in need thereof, said method comprising:
obtaining a sample of prostate tissue from the subject;
characterizing cancerous cells in the tissue sample to determine whether the cells possess a receptor for PDF;
characterizing the cells to determine whether the cells synthesize and secrete a precursor of PDF; and
characterizing the cells to determine whether the cells process the precursor of PDF to produce active PDF.

10. A method in accordance with claim 9 wherein the cells do not synthesize and secrete the precursor of PDF, said method further comprising the step of administering to the subject a therapeutically effective amount of PDF.

11. A method in accordance with claim 10 wherein administering a therapeutically effective amount of active PDF comprises administering a therapeutically effective amount of the PDF in a pharmaceutically acceptable carrier.

12. A method in accordance with claim 9 wherein the cells do not synthesize and secrete the precursor of PDF, said method further comprising the step of administering to the subject an amount of the precursor of PDF together with an amount of proprotein convertase wherein together the amount of the precursor of PDF and the amount of proprotein convertase are sufficient to provide a therapeutically effective amount of PDF.

13. A method in accordance with claim 12 wherein administering an amount of the precursor of PDF and an amount of proprotein convertase comprises administering the amount of PDF and the amount of proprotein convertase together in single dosage form in a pharmaceutically acceptable carrier.

14. A method in accordance with claim 12 wherein administering an amount of the precursor of PDF and an amount of proprotein convertase comprises administering the amount of the PDF in a pharmaceutically acceptable carrier in a first dosage form, and administering the amount of proprotein convertase in a pharmaceutically acceptable carrier in a second dosage form separate from the first dosage form.

15. A method in accordance with claim 9 wherein the cells synthesize and secrete the precursor of PDF but do not process the precursor of PDF, said method further comprising the step of administering to the subject a therapeutically effective amount of PDF.

16. A method in accordance with claim 15 wherein administering a therapeutically effective amount of PDF comprises administering a therapeutically effective amount of the PDF in a pharmaceutically acceptable carrier.

17. A method in accordance with claim 9 wherein the cells synthesize and secrete a precursor of PDF but do not process the precursor of PDF, said method further comprising the step of administering to the subject a therapeutically effective dose of a proprotein convertase for processing the precursor of PDF.

18. A method in accordance with claim 17 wherein administering a therapeutically effective amount of a proprotein convertase comprises administering a therapeutically effective amount of the proprotein convertase in a pharmaceutically acceptable carrier.

19. A method for treating prostate cancer in a subject in need thereof, said method comprising promoting prostate cell differentiation in the subject by administering to the subject an agent for increasing the biological activity of PDF in the subject at an early stage of the prostate cancer.

20. A method in accordance with claim 19 wherein administering to the subject an agent for increasing the biological activity of PDF in the subject comprises administering to the subject a therapeutically effective amount of PDF.

21. A method in accordance with claim 20 wherein administering a therapeutically effective amount of PDF comprises administering a therapeutically effective amount of the PDF in a pharmaceutically acceptable carrier.

22. A method in accordance with claim 19 wherein administering to the subject an agent for increasing the biological activity of PDF in the subject comprises administering to the subject an amount of a precursor of PDF and an amount of proprotein convertase wherein together the amount of the precursor of PDF and the amount of proprotein convertase are sufficient to provide a therapeutically effective amount of PDF.

23. A method in accordance with claim 22 wherein administering an amount of a precursor of PDF and an amount of proprotein convertase comprises administering the amount of PDF and the amount of proprotein convertase together in single dosage form in a pharmaceutically acceptable carrier.

24. A method in accordance with claim 22 wherein administering an amount of a precursor of PDF and an amount of proprotein convertase comprises administering the amount of the PDF in a pharmaceutically acceptable carrier in a first dosage form, and administering the amount of proprotein convertase in a pharmaceutically acceptable carrier in a second dosage form separate from the first dosage form.

25. A method in accordance with claim 19 wherein administering to the subject an agent for increasing the biological activity of PDF in the subject comprises administering to the subject a therapeutically effective amount of a proprotein convertase.

26. A method in accordance with claim 25 wherein administering a therapeutically effective amount of a proprotein convertase comprises administering a therapeutically effective amount of the proprotein convertase in a pharmaceutically acceptable carrier.

27. A composition for treating or preventing prostate cancer in a subject, said composition comprising a therapeutically effective amount of PDF in a pharmaceutically acceptable carrier.

28. A composition for treating or preventing prostate cancer in a subject, said composition comprising an amount of an inactive precursor of PDF and an amount of proprotein convertase in a pharmaceutically acceptable carrier, wherein together the amount of the inactive precursor of PDF and the amount of proprotein convertase are sufficient to provide a therapeutically effective amount of PDF.

29. A composition in accordance with claim 28 comprising the amount of inactive precursor of PDF, the amount of proprotein convertase and the pharmaceutically acceptable carrier in a single dosage form.

30. A composition in accordance with claim 28 comprising the amount of precursor of PDF in an amount of the pharmaceutically acceptable carrier in a first dosage form, and the amount of proprotein convertase in an amount of the pharmaceutically acceptable carrier in a second dosage form separate from the first dosage form.

31. A composition for treating or preventing prostate cancer in a subject, said composition comprising a therapeutically effective amount of proprotein convertase in a pharmaceutically acceptable carrier.
